(19) United States
(12) Patent Application Publication
(10) Pub. No.: US 2026/0337080 A1
(43) Pub. Date: Oct. 29, 2026

(54) SYSTEM AND METHOD FOR TREATMENT OF AN ORGANIC SUBSTRATE

(71) Applicants: Efim MONOSOV, River Hill (WI); ORGANIC GAS MANAGEMENT S.L., Ponferrada León (ES)

(72) Inventors: Efim MONOSOV, River Hill, WI (US); Elroy AMITZUR, Savyon (IL); Irina GOKHMAN, Milwaukee, WI (US); Yuri SHNOL, Bat Yam (IL)

(21) Appl. No.: 19/156,924

(22) PCT Filed: Feb. 22, 2024
(86) PCT No.: PCT/IB2024/051724

§ 371 Date: Aug. 15, 2025

Related U.S. Application Data
(60) Provisional application No. 63/447,534, filed on Feb. 22, 2023.

(51) Int. Cl. C02F 11/04; C02F 11/02; C02F 11/18; C02F 103/20; C02F 103/22; C02F 103/32

(52) U.S. Cl. CPC C02F 11/04; C02F 11/02; C02F 11/18; C02F 2103/20; C02F 2103/22; C02F 2103/32

(57) ABSTRACT

Systems and methods for pre-treating organic substrates so as to enhance the efficiency of anaerobic and aerobic digestion of the organic substrates, the system including: a conduit, adapted to have a stream flow therethrough, the stream containing the liquid medium and the organic substrate; an electromagnetic field generator, disposed circumferentially about the conduit, the electromagnetic field generator adapted to induce an electromagnetic field to an interior of the conduit, such that, when the liquid medium flows through the conduit, the electromagnetic field is applied to the liquid medium; and a microwave emitter, attached to the conduit, said microwave emitter adapted to emit microwave radiation into the conduit, such that, when the liquid medium flows through the conduit, the microwave radiation is applied to the liquid medium.

This application draws priority from US Provisional Patent Application No. U.S. 63/447,534, filed Feb. 23, 2023, which application is incorporated by reference for all purposes as if fully set forth herein.

### FIELD OF THE INVENTION

The present invention relates in general to systems and methods for processing or digesting of organic substrates, and more particularly, to systems and methods for pre-treating organic substrates to enhance the efficiency of anaerobic and aerobic digestion of the organic substrates.

### SUMMARY OF THE INVENTION

In accordance with an embodiment of the present invention, there is provided a system for improving digestion of an organic substrate in a liquid medium, the system including: a conduit, adapted to have a stream flow therethrough, the stream containing the liquid medium and the organic substrate; an electromagnetic field generator, disposed circumferentially about the conduit, the electromagnetic field generator adapted to induce an electromagnetic field to an interior of the conduit, such that, when the liquid medium flows through the conduit, the electromagnetic field is applied to the liquid medium; and a microwave emitter, attached to the conduit, the microwave emitter adapted to emit microwave radiation into the conduit, such that, when the liquid medium flows through the conduit, the microwave radiation is applied to the liquid medium.

In embodiments, the conduit is a pipe, adapted to be in fluid communication with a source of the liquid medium in an operational mode of the system.

In embodiments, the system further includes at least one valve disposed between the source of the liquid medium and the conduit, and in the operational mode of the system, the valve is open and allows the liquid medium to flow from the source to the conduit.

In embodiments, the system further includes a pump, and in the operational mode of the system, the pump is adapted to pump the liquid medium from the source to the conduit.

In embodiments, the conduit is a sleeve, adapted to be disposed about a pipe, the pipe adapted to be in fluid communication with a source of the liquid medium in an operational mode of the system.

In embodiments, the electromagnetic field generator includes a metal wire forming a spiral about the conduit, and adapted to generate the electromagnetic field when electricity passes through the metal wire.

In embodiments, the electromagnetic field generator is adapted to induce an electromagnetic field having a first frequency in the range of 5 Hz to 500 Hz.

In embodiments, the electromagnetic field generator is adapted whereby the electromagnetic field is an alternating current (AC) electromagnetic field.

In embodiments, the microwave emitter is adapted to emit microwave radiation having a second or microwave frequency in the range of 1 GHz to 20 GHz.

In embodiments, the microwave emitter includes a microwave generator and at least one antenna extending from the microwave generator into the conduit.

In embodiments, the system further includes a controller, functionally associated with the microwave emitter and with the electromagnetic field generator, the controller being adapted to electronically control or modulate operation of the electromagnetic field generator and of the microwave emitter, in accordance with at least one criterion of the conduit or of the liquid medium within the conduit.

In embodiments, the system further includes at least one sensor, functionally associated with the controller, and adapted to provide to the controller input relating to the at least one criterion.

In embodiments, the at least one sensor includes a flow rate sensor. In embodiments, the at least one sensor includes a temperature sensor. In embodiments, the at least one sensor includes a pH sensor. In embodiments, the at least one sensor includes a chemical sensor.

In embodiments, the conduit has a length in the range of 1 m to 5 m.

In embodiments, the conduit has an inside diameter in the range of 75 mm to 250 mm.

In embodiments, the electromagnetic field generator is disposed closer to an upstream end of the conduit than the microwave emitter, such that, when the liquid medium flows through the conduit, the electromagnetic field is applied to the liquid medium prior to the microwave radiation being applied to the liquid medium.

In embodiments, the electromagnetic field generator is disposed closer to a downstream end of the conduit than the microwave emitter, such that, when the liquid medium flows through the conduit, the electromagnetic field is applied to the liquid medium following the microwave radiation being applied to the liquid medium.

In embodiments, the electromagnetic field generator is configured to induce the electromagnetic field in a segment the conduit, and the microwave emitter is adapted to emit microwave radiation into the segment of the conduit, such that, when the liquid medium flows through the segment of the conduit, the liquid medium concurrently has the electromagnetic field and the microwave radiation applied thereto.

In embodiments, an output of the conduit is in fluid communication with a digester, such that following flowing through the conduit, the liquid medium reaches the digester. In embodiments, the digester is an anaerobic digester. In embodiments, the digester is an aerobic digester.

In embodiments, the system further includes the liquid medium including the organic substrate.

In embodiments, during passage through the conduit, a temperature of the liquid medium is in the range of 5 C to 60 C. In embodiments, during passage through the conduit, a temperature of the liquid medium is in the range of 15 C to 60 C. In embodiments, during passage through the conduit, a temperature of the liquid medium is in the range of 30 C to 55 C.

In embodiments, during passage through the conduit, a pH of the liquid medium is in the range of 3 to 11. In embodiments, during passage through the conduit, a pH of the liquid medium is in the range of 4 to 10. In embodiments, during passage through the conduit, a pH of the liquid medium is in the range of 5 to 10. In embodiments, during passage through the conduit, a pH of the liquid medium is in the range of 6 to 9.

In embodiments, the concentration of the organic substrate within the stream is within the range of 1% to 20%.

In embodiments, the organic substrate includes biomass.

In embodiments, the organic substrate includes complex organic polymers. In embodiments, the complex organic polymers include lignin. In embodiments, the complex organic polymers include cellulose. In embodiments, the complex organic polymers include pectin.

In embodiments, the organic substrate includes industrial waste. In some embodiments, the organic substrate includes sanitary waste. In embodiments, the organic substrate includes agricultural waste.

In accordance with an aspect of the present invention, there is provided a method for pre-treating an organic substrate in a liquid medium to facilitate anaerobic or aerobic digestion thereof, the method including: allowing a stream of the liquid medium including the organic substrate, to flow through a conduit; and during passage of the liquid medium through the conduit: inducing an electromagnetic field to the interior of the conduit, such that the electromagnetic field is applied to the liquid medium; and emitting microwave radiation into the conduit, such that the microwave radiation is applied to the liquid medium.

In accordance with another aspect of the present invention, there is provided a method of producing biogas from an organic substrate in a liquid medium, the method including: allowing a stream of the liquid medium including the organic substrate, to flow through a conduit; during passage of the liquid medium through the conduit: inducing an electromagnetic field to the interior of the conduit, such that the electromagnetic field is applied to the liquid medium; and emitting microwave radiation into the conduit, such that the microwave radiation is applied to the liquid medium; and producing biogas by digestion of the liquid medium exiting the conduit, following the liquid medium having had the electromagnetic field and the microwave radiation applied thereto.

In embodiments, the allowing the liquid medium to flow through the conduit includes opening a valve between the conduit and a source of the liquid medium to allow the liquid medium to flow into the conduit.

In embodiments, the allowing the liquid medium to flow through the conduit includes pumping the liquid medium into the conduit.

In embodiments, the inducing the electromagnetic field includes passing electricity through a metal wire forming a spiral about the conduit.

In embodiments, the electromagnetic field generator is adapted to induce an electromagnetic field having a first frequency in the range of 5 Hz to 500 Hz.

In embodiments, the inducing the electromagnetic field includes inducing an alternating current (AC) electromagnetic field.

In embodiments, the microwave emitter is adapted to emit microwave radiation having a second or microwave frequency in the range of 0.5 GHz to 20 GHz. In embodiments, the method further includes electronically controlling or modulating the inducing of the electromagnetic field generator and the emitting of the microwave radiation, in accordance with at least one criterion of the conduit or of the liquid medium within the conduit.

In embodiments, the at least one criterion includes a flow rate of the liquid medium within the conduit. In some embodiments, the at least one criterion includes a temperature of the liquid medium within the conduit. In some embodiments, the at least one criterion includes a temperature of the conduit. In some embodiments, the at least one criterion includes a pH of the liquid medium within the conduit.

In embodiments, the controlling includes determining whether the at least one criterion is met based on at least one sensor input.

In embodiments, the at least one sensor input includes input from a flow rate sensor. In some embodiments, the at least one sensor input includes input from a temperature sensor. In some embodiments, the at least one sensor input includes input from a pH sensor. In some embodiments, the at least one sensor input includes input from a chemical sensor.

In embodiments, the flow rate is in the range of 1 cm per second to 10 cm per second.

In embodiments, the inducing the electromagnetic field occurs prior to the emitting the microwave radiation, such that, when the liquid medium flows through the conduit, the electromagnetic field is applied to the liquid medium prior to the microwave radiation being applied to the liquid medium.

In embodiments, the inducing the electromagnetic field occurs following the emitting the microwave radiation, such that, when the liquid medium flows through the conduit, the electromagnetic field is applied to the liquid medium following the microwave radiation being applied to the liquid medium.

In embodiments, the inducing the electromagnetic field occurs concurrently with the emitting the microwave radiation, such that, when the liquid medium flows through the segment of the conduit, the liquid medium concurrently has the electromagnetic field and the microwave radiation applied thereto.

In embodiments, the method further includes delivering the output of the conduit to an anaerobic digester for anaerobic digestion of the organic substrate in the liquid medium.

In embodiments, the method further includes delivering the output of the conduit to an aerobic digester for aerobic digestion of the organic substrate in the liquid medium.

In embodiments, a temperature of the liquid medium is in the range of 5 C to 60 C.

In embodiments, during passage through the conduit, a pH of the liquid medium is in the range of 3 to 11.

In accordance with a further aspect of the present invention, there is provided a method of retrofitting an anaerobic digestion plant to pre-treat organic substrate to be anaerobically digested, the anaerobic digestion plant including an input conduit leading the organic substrate to an anaerobic digester, the method including placing a pre-treatment sleeve about the input conduit, the pre-treatment sleeve including: an electromagnetic field generator, disposed circumferentially about the pre-treatment sleeve; and a microwave emitter, disposed on or in the pre-treatment sleeve, wherein, during flow of a liquid medium including the organic substrate through the input conduit: the microwave emitter is adapted to emit microwave radiation into the conduit, such that, when the liquid medium flows through the input conduit, the microwave radiation is applied to the liquid medium, and the electromagnetic field generator is adapted to induce an electromagnetic field in interior of the input conduit, such that, when the liquid medium flows through the input conduit, the electromagnetic field is applied to the liquid medium.

In accordance with another aspect of the present invention, there is provided a method of retrofitting an aerobic wastewater treatment plant to pre-treat biosolids to increase its bio-degradability, the aerobic wastewater treatment plant including an input conduit leading the organic substrate to an aerobic digester, the method including placing a pre-treatment sleeve about the input conduit, the pre-treatment sleeve including: an electromagnetic field generator, disposed circumferentially about the pre-treatment sleeve; and a microwave emitter, disposed on or in the pre-treatment sleeve,
wherein, during flow of a liquid medium including the organic substrate through the input conduit: the microwave emitter is adapted to emit microwave radiation into the conduit, such that, when the liquid medium flows through the input conduit, the microwave radiation is applied to the liquid medium, and the electromagnetic field generator is adapted to induce an electromagnetic field in interior of the input conduit, such that, when the liquid medium flows through the input conduit, the electromagnetic field is applied to the liquid medium.

### BRIEF DESCRIPTION OF THE FIGURES

The foregoing discussion will be understood more readily from the following detailed description of the invention, when taken in conjunction with the accompanying FIGS. 1A to 4), in which:

FIGS. 1A, 1B, and 1C are schematic illustrations of three configurations of a system for pre-treating an organic matter in a liquid medium, so as to improve digestion of the organic matter, according to embodiments of the disclosed technology;

FIG. 2 is a schematic illustration of a configuration of a system for pre-treating an organic matter in a liquid medium, suitable for retrofitting onto an existing pipe in a facility for digestion of organic matter, according to embodiments of the disclosed technology;

FIGS. 3A, 3B, and 3C are schematic illustrations of three configurations of an anaerobic digestion systems for digestion of organic matter in a liquid medium, each of the anaerobic digestion systems including the system of any one of FIGS. 1A to 2, according to embodiments of the disclosed technology; and

FIG. 4 is a flow chart of a method of using the system of any one of FIGS. 1A to 2, to pre-treat organic substrate in a liquid medium to facilitate anaerobic digestion of the organic substrate, according to embodiments of the disclosed technology.

### DESCRIPTION OF THE PREFERRED EMBODIMENTS

The principles of the inventive system and method for improving digestion of an organic matter in a liquid medium by pre-treating the organic matter, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

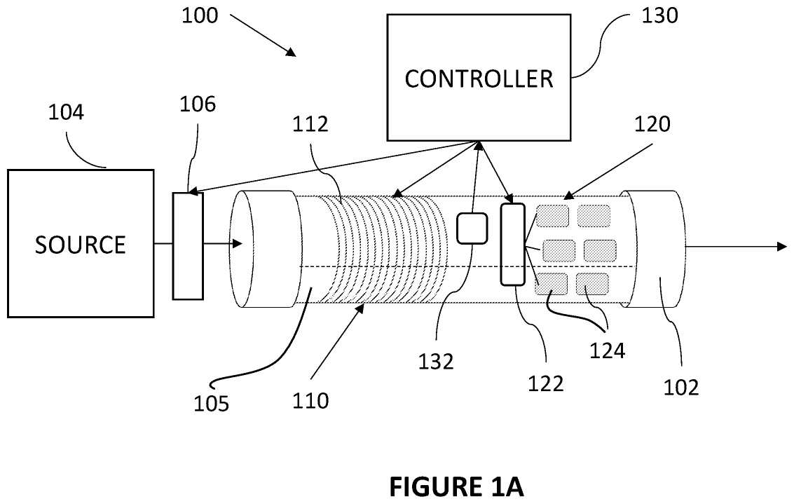

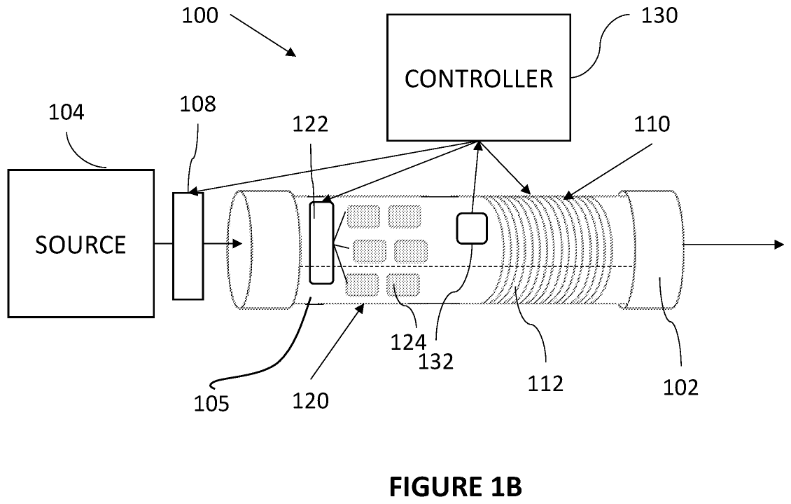

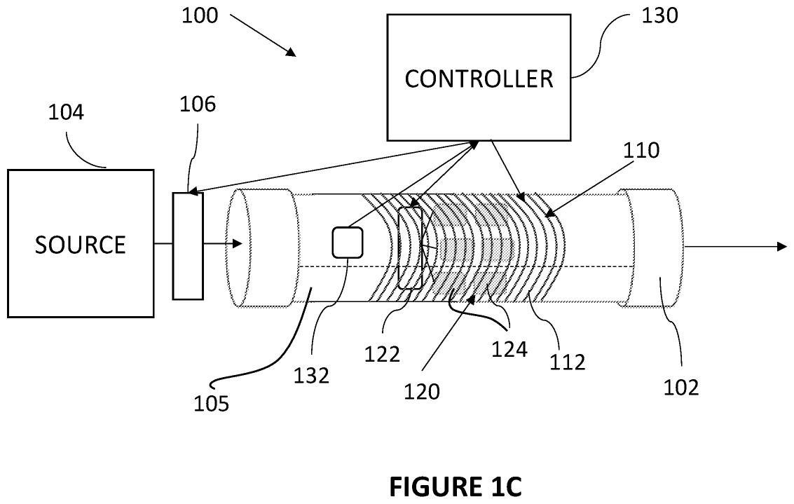

Referring now to the drawings, FIGS. 1A, 1B, and 1C are schematic illustrations of three configurations of a system 100 for pre-treating an organic matter in a liquid medium, so as to improve digestion of the organic matter, according to embodiments of the disclosed technology.

As seen in FIGS. 1A, 1B, and 1C, system 100 includes a conduit 102, which is typically in fluid communication with a source 104 of a liquid medium. In an operative state of system 100, conduit 102 has a stream 105 flow therethrough, from the source 104, the stream including the liquid medium and the organic matter. For example, the operative mode may be facilitated by opening of a valve 106 (shown clearly in FIG. 1A) allowing the liquid medium and organic matter to flow out of source 104 into conduit 102, to form the stream. As another example, a pump 108 (shown clearly in FIG. 1B) may be operated to pump the liquid medium and the organic matter from source 104 to conduit 102, to form the stream 105.

In some embodiments, conduit 102 has a length in the range of 1 m to 3 m. In some embodiments, conduit 102 has a length in the range of 1.5 m to 2.5 m. In some embodiments, conduit 102 has a length of 2 m.

In some embodiments, conduit 102 has an inside diameter in the range of 75 mm to 250 mm, 75 mm to 200 mm, 75 mm to 150 mm, 100 mm to 250 mm, 100 mm to 2000 mm, or 100 mm to 150 mm.

System 100 further includes an electromagnetic field generator 110, disposed circumferentially about conduit 102. Electromagnetic field generator 110 is adapted to induce an electromagnetic field to the interior of conduit 102, such that, when the liquid medium flows through the conduit, the induced electromagnetic field is applied to the flowing liquid medium. Electromagnetic field generator 110 may be any suitable electromagnetic field generator. However, in FIGS. 1A to 1C, the electromagnetic field generator is implemented as a metal wire 112, for example a copper wire, wound in a spiral about conduit 102. Wire 112 is adapted to generate the electromagnetic field when electricity passes therethrough. Typically, electromagnetic field generator 110 is such that the generated electromagnetic field is an alternating current (AC) electromagnetic field.

In some embodiments, electromagnetic field generator 110 is adapted to induce an electromagnetic field having a first frequency in the range of 5 Hz to 500 Hz. More typically, the first frequency is at least 10 Hz, at least 20 Hz, at least 25 Hz, at least 30 Hz, at least 35 Hz, or at least 40 Hz. More typically, the first frequency is at most 400 Hz, at most 300 Hz, at most 250 Hz, at most 200 Hz, at most 150 Hz, at most 125 Hz, at most 100 Hz, at most 80 Hz, at most 70 Hz, or at most 60 Hz.

System 100 further includes a microwave emitter 120, attached to conduit 102. Microwave emitter 120 is adapted to emit microwave radiation into conduit 102, such that, when the liquid medium flows through the conduit, the emitted microwave radiation is applied to the liquid medium. In some embodiments, microwave emitter 120 may be attached to an exterior surface of conduit 102. In some embodiments, at least a portion of microwave emitter 120 may be disposed within conduit 102.

In some embodiments, microwave emitter 120 may include a magnetron 122, and at least one antenna 124 extending from the magnetron to conduit 102. In some embodiments, magnetron 122 may be attached to an exterior of conduit 102, while antenna(s) 124 may extend into an interior of the conduit.

In some embodiments, microwave emitter 120 is adapted to emit microwave radiation having a second or microwave frequency in the range of 1 GHz to 20 GHz, 1 GHz to 18 GHz, 1 GHz to 12 GHz, 1 GHz to 10 GHz, 1 GHz to 8 GHz, 1 GHz to 6 GHz, 1 GHz to 5 GHz, 1 GHz to 4 GHz, 1 GHz to 3 GHz, 1.25 GHz to 3 GHz, 1.5 GHz to 3 GHz, 1.5 GHz to 2.75 GHz, 1.5 GHz to 2.5 GHz, or 1.75 GHz to 2.25 GHz.

In some embodiments, system 100 may further include a controller 130, functionally associated with electromagnetic field generator 110 and with microwave emitter 120. Controller 130 is adapted to electronically control or modulate operation of electromagnetic field generator 110 and of microwave emitter 120 based on various criteria of their operation, such as a flow rate of the liquid medium within conduit 102, a temperature of the liquid medium in conduit 102 or of the conduit itself, a pH of the liquid medium in conduit 102, and the like. In some embodiments, controller 130 may further be associated with at least one sensor 132, disposed in or on conduit 102. Sensor(s) 132 may provide input to controller 130 regarding parameters of the liquid medium or of conduit 102. For example, sensor(s) 132 may be, or include, a flow rate sensor, a temperature sensor, or a pH sensor.

In the operative state of system 100, liquid medium that passed through conduit 102 is output downstream for further processing, as explained in further detail hereinbelow.

Turning now specifically to FIG. 1A, it is seen that electromagnetic field generator 110 is disposed at, or near, an upstream end of conduit 102, close to source 104, and microwave emitter 120 is disposed at, or near, a downstream end of conduit 102. As such, when liquid medium flows through conduit 102, the electromagnetic field is applied to the liquid medium prior to the microwave radiation impacting the liquid medium.

In FIG. 1B, electromagnetic field generator 110 is disposed at, or near, an upstream end of conduit 102, close to source 104, and microwave emitter 120 is disposed at, or near, a downstream end of conduit 102. As such, when liquid medium flows through conduit 102, the microwave radiation impacts the liquid medium prior to the electromagnetic field being applied to the liquid medium.

In FIG. 1C, electromagnetic field generator 110 induces an electromagnetic field in a segment 102a of conduit 102, and microwave emitter 120 emits microwave radiation into the same segment of conduit 102. As such, when liquid medium flows through segment 102a, the liquid medium is concurrently impacted by the electromagnetic field and by the microwave radiation.

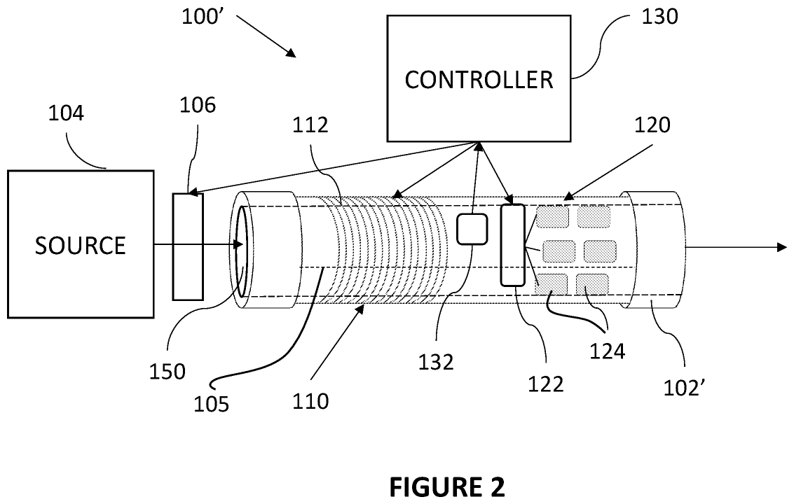

Reference is now made to FIG. 2, which is a schematic illustration of a configuration of a system 100′ for pre-treating an organic matter in a liquid medium. System 100′ is suitable for retrofitting onto an existing pipe in a facility for digestion of organic matter, according to embodiments of the disclosed technology.

System 100′ of FIG. 2 is substantially similar to system 100 of FIGS. 1A to 1C, with like reference numerals indicating like components. However, in system 100′, conduit 102′ is a sleeve, adapted to be wrapped around an existing pipe 150 in a facility for digestion of organic matter, such as an anaerobic digestion facility. As such, in some embodiments, conduit 102′ may include a longitudinal slot, facilitating wrapping of pipe 150 with conduit 102′. In some other embodiments, conduit 102′ may be a complete cylinder, and may be slid onto pipe 150 from a longitudinal end of pipe 150. For example, conduit 102′ may be placed by disconnecting pipe 150 from pipes or receptacles communicating with it on upstream and downstream ends thereof, sliding conduit 102′ onto pipe 150, and then reconnecting the conduit to is neighboring pipes or receptacles.

In some embodiments, in which antenna(s) 124 are to be disposed within the liquid medium to be processed, installation of conduit 102′ onto pipe 150 may include inserting antenna(s) 124 into an interior of pipe 150.

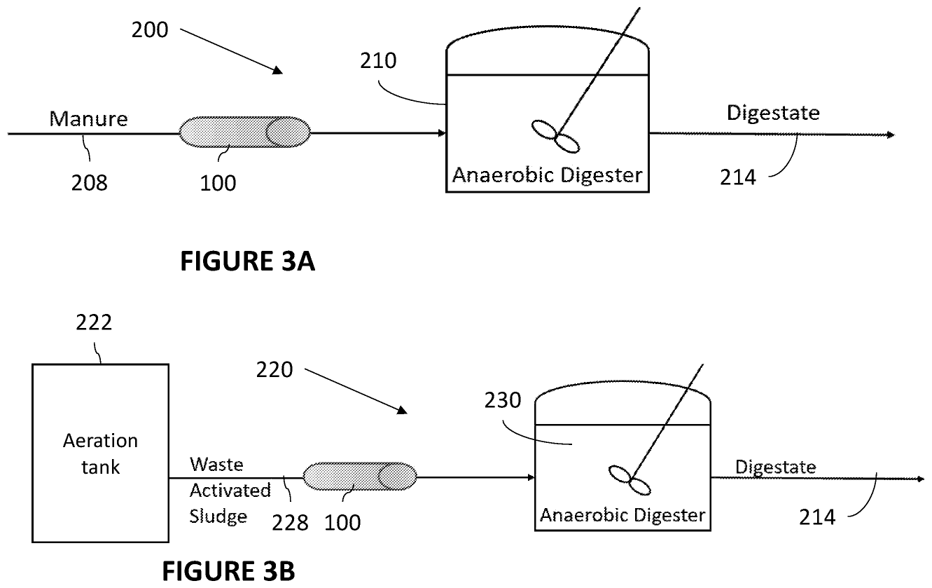

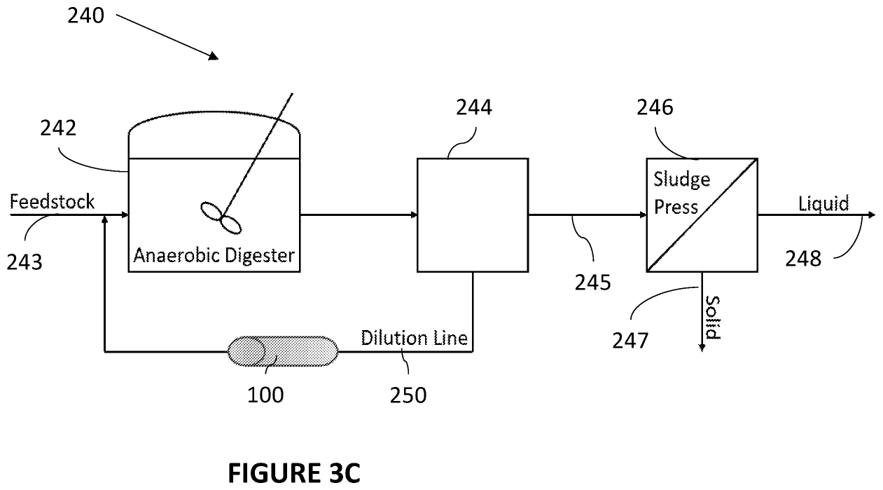

Reference is now made to FIGS. 3A, 3B, and 3C, which are schematic illustrations of three configurations of an anaerobic digestion system for digestion of organic matter in a liquid medium. Each of the anaerobic digestion systems includes system 100 of any one of FIGS. 1A to 1C or system 100′ of FIG. 2, according to embodiments of the disclosed technology.

As seen in FIG. 3A, a system 200 may be, or form part of, a sanitation plant, which digests organic substrates originating from manure. As seen, system 100 is applied to an inlet line 208, which feeds a liquid medium including manure into an anaerobic digester 210. As such, the liquid medium flowing toward the anaerobic digester undergoes application of the electromagnetic field and the microwave radiation thereto. In anaerobic digester 210 the organic substrate is further digested, for example to form biogas. The resulting digestate is removed from the anaerobic digester 210, as indicated by arrow 214, and the resulting biogas flows out of anaerobic digester 210 into a suitable tank, as known in the art.

Turning to FIG. 3B, a system 220 may be, or form part of, a wastewater digestion plant, which digests organic substrates originating from waste water, such as those found in municipal waste systems. Typically, the wastewater initially undergoes an activated sludge process, in which the wastewater is turned into waste activated sludge or return activated sludge, which is a liquid solution of organic substrates, typically including biomass. For example, the wastewater may be aerated in a suitable tank 222. System 100 (or 100′) is applied to an inlet line leading from tank 222 to an anaerobic digester 230. As such, the liquid medium flowing from tank 222 toward anaerobic digester 230 undergoes application of the electromagnetic field and the microwave radiation thereto. In anaerobic digester 230 the organic substrate is further digested, for example to form biogas. The digestate biogas, is removed from the anaerobic digester 230, as indicated by arrow 234, and the resulting biogas flows out of anaerobic digester 230 into a suitable tank, as known in the art.

In FIG. 3C, a system 240 may be, or form part of, an agricultural digestion plant, which digests organic substrates originating from animal feedstock. System 240 differs from systems 200 and 220 in that it may be cyclic, such that some organic substrate may undergo digestion more than once. As seen in FIG. 3C, organic feedstock, typically in a liquid medium, is fed into an anaerobic digester 242 via an inlet pipe 243. The output of anaerobic digester 242 is provided to digestate buffer tank 244, for further processing.

A first portion of the output of digestate buffer tank 244, indicated by arrow 245 is fed into a sludge press 246, which separates the processed liquid from the processed solid. The processed solids, also known as biosolids, are removed from sludge press 246 for further use, for example in application to fields for providing organic matter to the soil and for improvement of the crops, as indicated by arrow 247. The processed liquid is removed from sludge press 246 and may be reused, or safely disposed of, as indicated by arrow 248.

A second portion of the output of digestate buffer tank 244 flows into a dilution line 250, onto which system 100 (or 100′) is applied, for application of the electromagnetic field and the microwave radiation to liquid medium in the dilution line. The pre-treated liquid medium in the dilution line delivered back to anaerobic digester 242, or may be merged with an inlet line prior to delivery to the anaerobic digester. As mentioned above, in the anaerobic digester, the organic substrate is further digested, for example to form biogas, as known in the art.

It is appreciated that in some applications, system 100 may be applied to inlet pipe 243, for pre-treatment of the initial, undiluted, solution. The effectiveness of application of system 100 to inlet pipe 243 depends on the solid content, or dry matter content, within the liquid medium.

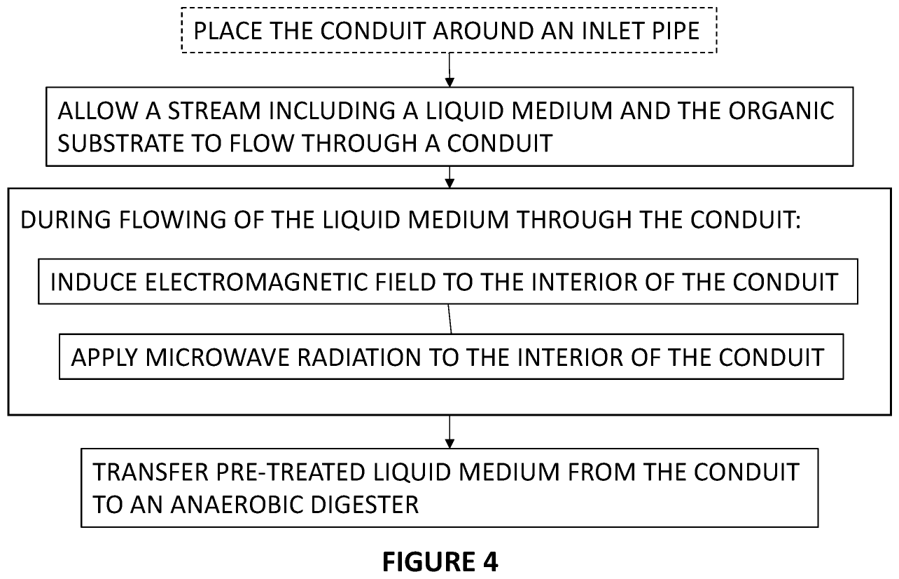

Reference is now additionally made to FIG. 4, which is a flow chart of a method of using the system 100 of any one of FIGS. 1A to 1C or the system 100′ of FIG. 2, to pre-treat an organic substrate in a liquid medium to facilitate anaerobic digestion of the organic substrate, according to embodiments of the disclosed technology.

As seen at step 300, a stream including the liquid medium including the organic substrate is introduced, or allowed to flow into, and through, conduit 102 (FIGS. 1A to 1C) or pipe 150 circumscribed by conduit 102′ (FIG. 2).

In some embodiments, a concentration of the organic substrate within the stream is within the range of 1% to 20%, 3% to 15%, 3% to 12%, 5% to 12%, or 5% to 10%.

In some embodiments, the concentration of the organic substrate within the stream is at least 2.5%, at least 3.5%, or at least 4%.

In some embodiments, the concentration of the organic substrate within said stream is at most 16%, at most 14%, at most 12%, at most 10%, at most 8%, or at most 7%.

As discussed hereinabove, in some embodiments, the organic substrate comprises waste materials, and may include any one or more of industrial waste, sanitary waste, and agricultural waste. For example, industrial waste may include waste from paper processing, beer manufacturing, food manufacturing, or tannery plants.

In some embodiments, the organic substrate comprises biomass and biosolids. Typically, the biomass and biosolids aid in the anaerobic digestion process.

In some embodiments, the organic substrate comprises complex organic polymers that are difficult to biodegrade, such as lignin cellulose, and pectin.

In some embodiments, during flowing of the liquid medium through conduit 102 or 102′, the liquid medium has a temperature in the range of 5 C to 60 C, 10 C to 60 C, 15 C to 60 C, 20 C to 60 C, 25 C to 60 C, 25 C to 55 C, or 30 C to 55 C. It is to be appreciated that temperatures outside of these ranges may harm, or even kill, the biomass (e.g. bacteria) within the organic substrate, which would limit, slow, or stop the anaerobic digestion of the organic substrate, following its passage in the conduit.

In some embodiments, during flowing of the liquid medium through conduit 102 or 102′, the liquid medium has a pH in the range of 6 to 9. It is to be appreciated that a pH outside of this range may harm, or even kill, the biomass (e.g. bacteria) within the organic substrate, which would limit, slow, or stop the anaerobic digestion of the organic substrate, following its passage in the conduit.

In some embodiments, the flow rate of the liquid medium through conduit 102 or 102′ is in the range of 1 cm/second to 10 cm/second. The dimensions of conduit 102 or 102′, discussed hereinabove with respect to FIGS. 1A to 1C, are selected to facilitate the required flow rate, while at the same time minimizing energy consumption by the pump, or other mechanism, providing the liquid medium to conduit 102.

In some embodiments, the flow time of the liquid medium through the entire conduit is not more than 15 minutes, 10 minutes, 8 minutes, 6 minutes, 4 minutes, or 2 minutes. In some embodiments, the flow time of the liquid medium through the entire conduit is in the range of 15 seconds to 15 minutes, 15 seconds to 10 minutes, 15 seconds to 8 minutes, 15 seconds to 6 minutes, 15 seconds to 4 minutes, 15 seconds to 2 minutes, 30 seconds to 2 minutes, 30 seconds to 90 seconds, or 30 seconds to 1 minute.

It is to be appreciated that the short flow times through conduit 102 or 102′ are required in order to ensure that the application of the electromagnetic field and microwave radiation to the liquid medium does not raise the temperature of the liquid medium to be outside the working ranges listed above, and to ensure continued effectiveness of the anaerobic digestion process. In some embodiments, the temperature elevation may be held under 5 C, under 3 C, or under 2 C.

As seen at step 302, several steps take place during passage of the liquid medium through conduit 102 or 102′.

At step 304, an electromagnetic field is induced to the interior of conduit 102 or 102′, for example by electromagnetic field generator 110. The induced electromagnetic field impacts the liquid medium flowing through the conduit. When the liquid medium including the organic substrate flows through the electromagnetic field, and electrochemical current is also generated within the liquid medium.

At step 306, microwave radiation is emitted into the conduit 102 or 102′, for example by microwave emitter 120. The emitted microwave radiation is applied to the liquid medium flowing through the conduit.

In some embodiments, step 304 may occur prior to step 306. In some embodiments, step 306 may occur prior to step 304. In some embodiments, steps 304 and 306 may occur at least partially concurrently.

Typically, following passage of the liquid medium through conduit 102 or 102′ the pre-treated liquid medium, which was exposed to the electromagnetic field and to the microwave radiation, is transferred to an anaerobic digester for further digestion thereof, at step 308.

In some embodiments, such as embodiments in which conduit 102′ is used, the method includes an initial set-up step 310, in which the conduit 102′ is placed about pipe 150, as described hereinabove. In such embodiments, step 310 occurs prior to any of steps 300, 302, 304, 306, and 308.

In experimental results, the inventors have found that use of the method of FIG. 4 to pre-treat the liquid medium prior to anaerobic digestion thereof increases the biodegradability of the organic substrate in the liquid medium by at least 5%. In some applications, the biodegradability of the organic substrate increased from 80% (when simply using anaerobic digestion) to 85% (when using the method of FIG. 4). In some other applications, the biodegradability of the organic substrate increased from 70% (when simply using anaerobic digestion) to 90% (when using the method of FIG. 4). The improvement of the biodegradability of the organic substrate is beneficial for at least one of several reasons:
increases the amount of bio-methane that can be generated from the organic substrate;
reduces the retention time required for processing of the organic substrate, and as a result also reduces the amount of energy required for the process;
increases the capacity of digesters and, as a result, increases the profitability of the entire system;
reduces the waste remaining at the end of the anaerobic digestion; and
improves the quality of the effluent collected at the end of the anaerobic digestion process.

The inventors have surprisingly found that a combination of application of an electromagnetic field and of microwave energy to the liquid medium, as in the method of FIG. 4, is energetically more efficient than the equivalent application of only one of these methodologies.

Thus, the inventors have surprisingly found that the combination of an electromagnetic field with application of microwave energy has a synergistic effect, resulting in a significant decrease in the energy required for pre-treatment of the liquid medium using the two methodologies together, as compared to using only one of the two methodologies to pre-treat the liquid medium. In order to achieve the same conversion, the energy consumption of the method of the present invention is appreciably lower than the energy consumption when using only application of an electromagnetic field or only application of microwave energy.

The inventors have also surprisingly discovered that application of microwave energy to the liquid medium while the liquid medium is flowing through the electromagnetic field does not inhibit, or reduce, the impact of the electromagnetic field on the liquid medium. Additionally, the electrochemical effect of the solution flowing through the electromagnetic field also remains unharmed, and in some cases may even become more effective, by simultaneous application of microwave energy.

### EXAMPLES

Reference is now made to the following examples, which together with the above descriptions, illustrate the invention in a non-limiting fashion.

### Example 1: Substrates

The laboratory experiments were carried out on the following substrate feedstocks:
Pig manure
Dairy Manure
Biosolids from municipal wastewater treatment plant
FOG (Grease Trap Waste)
DAF sludge from poultry slaughterhouse wastewater treatment plants
Brewer's Spent Grain (ground)
Ground Treated Seeds
Corn Silage
   Samples of the brewer's spent grain, ground treated seeds, and corn silage were diluted to a concentration of 3-5% of DM before treatment by MW & EMF.

### Example 2: Biomethane Potential Test (BMP)

The BMP tests were performed according to ISO 15985:2014, which specifies a method for the evaluation of the ultimate anaerobic biodegradability of plastics based on organic compounds under high-solids anaerobic-digestion conditions by measurement of evolved biogas at the end of the test. This method is designed to simulate typical anaerobic digestion conditions for the organic fraction of mixed municipal solid waste.

The BMP tests were used to determine biogas yield, methane concentration, and methane yield. Chemical parameters of the raw and treated substrate were analyzed in accordance with procedures under the Standard Methods for the Examination of Water and Wastewater of the American Water Works Association (AWWA).

After the addition of treated substrate samples, the batch bottles were flushed with N₂ for 2 minutes and 2 mL of saline solution (containing NH₄Cl, NaCl, CaCl₂·2H₂O and MgCl₂·6H₂O), 20 mL of inoculum as well as 20 mL of milli-Q water were added, then sealed with EPDM rubber stoppers and aluminum screw caps. The headspace of the batch bottles was exchanged with a gas mixture of CO₂ and N₂, thereafter 0.3 ml of Na₂S·3H₂O (100 mM) were added by means of a syringe.

The treatment combination and controls were performed in triplicate. All the samples were incubated at 37 °C. During the incubation, the biogas production and the methane content were measured on days 1, 3, 7, 12, 20, 28, and 35. The biogas production was measured prior to the methane analysis using a Testo 312-3 digital pressure gauge. After this analysis, 1 mL of headspace gas was extracted with a syringe and transferred into an 11 mL glass vial filled with air. From this vial, 1 mL was extracted and the methane content were analyzed in a Gas Chromatography Flame Ionization Detector (GC-FID), from the HP58880A series using a Poraplot T column and N₂ as the carrier gas (130 mL min⁻¹). The injector and detector temperatures were 150 °C and 250 °C, respectively. The detector gases were H₂ (30 mL min⁻¹) and air (250 mL min⁻¹). The methane content was calculated by fitting the measured values to a linear regression of the GC-FID readings of three controls.

The prepared samples were analyzed on a GC-FID (Clarus 580 Perkin-Elmer) having a BP21 column, helium as the carrier gas (2 mL min⁻¹), with a 1:10 split ratio injection (1 μL of sample). The injector and detector temperatures were 250 °C and 280 °C, respectively. The detector gases were H₂ (at 45 mL min⁻¹) and air (at 450 mL min⁻¹). The oven temperature was linearly ramped from 160 °C to 225 °C over 15 minutes, and subsequently ramped to 240 °C over 3 minutes.

With regard to the kinetic analysis, the degradation rate of the BMP was calculated using equation (1),

Y(t)=Ym·e^{-kt} (1)

wherein Y(t) is the accumulative methane yield (NmL g VS⁻¹) at day t, Ym is the total methane production (NmL g VS⁻¹), and k is the degradation rate (day⁻¹).

### Example 3: Semi-Continuous Digestion Experiments (SCADE)

The AD process was operated under mesophilic condition in a five-liter glass continuous stirred tank reactor (CSTR) having a working volume of 4 L. The mixing of the digester was facilitated by a mechanical stainless-steel stirrer operating at a constant rate. The SCADE utilized a set of two digesters: one control and one digester. The inoculum of the digesters was digestate from WWTP.

During the SCADE, volatile fatty acid (VFA) analyses were conducted on the control and on the MW & EMF treated digester. The VFA test monitored the changes in the concentrations of the VFAs prior to and after (8 h) the feeding.

Analyses were made on the digestate on a weekly basis. The digestate was analyzed for pH, conductivity, total solids and Volatile solid contents, ammonia, VFA and long chain fatty acid (LCFA) content.

Biogas production was measured daily using a custom-made gas meter. Collected overnight gas samples were analyzed weekly using a Biogas 5000 Geotech device, to determine biogas composition.

### Examples 4-11

Experiments were conducted on the feedstocks of Example 1. These feedstocks include a wide range of microorganisms responsible for the subsequent conversion into biogas and are representative of the organic waste produced and/or managed by agricultural and wastewater treatment operations-relevant to the majority of our target customers.

Demonstrating the performance and cost-effectiveness synergies resulting from combined MW/EMF treatment, we found that substrate samples subjected to MW+EMF treatment can increase methane yield by up to 20% at operating costs on the scale of $0.6 per pound of organic dry matter. In comparison, samples treated with MW or EMF alone increased methane yield by no more than 2-3% at a projected operating cost of $1.6 per pound of organic dry matter (oDM).

Subsequent experiments involved treatment of different types of organic feedstock: mixed with anaerobic biomass as an inoculum for the anaerobic fermentation process. The data presented in Table 1 are the average results of for experiments conducted on each type of feedstock, with each experiment being repeated at least 6 times, including a control or blank (untreated) test.

TABLE 1

| | | total dry | | Biogas | % | Biogas | Methane | % |
|---|---|---|---|---|---|---|---|---|
| | | matter in | ratio of | yield, | Methane | yield | yield, | Increase |
| | | feedstock | oDM to | l/kg raw | in | l/kg | l/kg raw | in CH₄ |
| Type of Feedstock | | (DM), % | DM, % | feedstock | biogas | oDM | feedstock | produced |
| Pig manure | Untreated | 5.8 | 76 | 12.74 | 56% | 289 | 7.13 | — |
| | Treated | | | 13.3 | 62% | 302 | 8.25 | 15.7% |
| Dairy manure | Untreated | 8.6 | 79 | 18.51 | 58% | 276 | 10.74 | — |
| | Treated | | | 20.25 | 64% | 298 | 12.96 | 20.67% |
| Concentrated | Untreated | 4.2 | 76 | 5.78 | 57% | 184 | 3.35 | — |
| biosolids | Treated | | | 6.41 | 61% | 201 | 3.91 | 16.7% |
| FOG (Grease | Untreated | 8.2% | 92% | 74.23 | 65% | 984 | 48.25 | — |
| Trap Waste) | Treated | | | 85.70 | 68% | 1,136 | 57.93 | 20.07% |
| DAF Sludge | Untreated | 14.2% | 90% | 91.25 | 58% | 714 | 52.92 | — |
| | Treated | | | 101.22 | 63% | 792 | 63.77 | 20.49% |
| Brewer's | Untreated | 18.2% | 80% | 72.22 | 56% | 496 | 40.44 | — |
| Spent Grain | Treated | | | 77.46 | 62% | 532 | 48.02 | 18.75% |
| Ground | Untreated | 93.1% | 96% | 669.43 | 59% | 749 | 394.96 | — |
| Treated | Treated | | | 745.40 | 63% | 834 | 469.60 | 18.90% |
| Seeds | | | | | | | | |
| Corn Silage | Untreated | 34.2% | 92% | 197.28 | 56% | 627 | 110.48 | — |
| | Treated | | | 214.90 | 61% | 683 | 131.95 | 19.44% |

The laboratory investigation demonstrated that the combination of MW radiation and EMF treatment provides an increase in methane production by >20%, due to an increase in biogas yield and methane concentration. The improvements in biogas yield, methane concentration (i.e., methane in biogas, %), and methane yield were found to be statistically significant (p<0.05) for each of the eight feedstock types tested.

Superior results were obtained within the following operating ranges: hydraulic retention time—30 to 60 seconds; alternating current frequency—30 to 120 Hz; electromagnetic flux density-1 to 3.5 mT; microwave frequency-1.5 to 4 GHz.

Typical electricity consumption was about 50 W*h per pound of oDM in the treated feedstock.

### Additional Embodiments

Additional Embodiments 1 to 174 are provided hereinbelow.

1. A system for improving digestion of an organic substrate in a liquid medium, the system comprising: a conduit, adapted to have a stream flow therethrough, said stream containing the liquid medium and the organic substrate; an electromagnetic field generator, disposed circumferentially about said conduit, said electromagnetic field generator adapted to induce an electromagnetic field to an interior of said conduit, such that, when the liquid medium flows through said conduit, said electromagnetic field is applied to said liquid medium; and a microwave emitter, attached to said conduit, said microwave emitter adapted to emit microwave radiation into said conduit, such that, when the liquid medium flows through said conduit, said microwave radiation is applied to the liquid medium.
2. The system of Embodiment 1, wherein said conduit is a pipe, adapted to be in fluid communication with a source of the liquid medium in an operational mode of said system.
3. The system of Embodiment 2, further comprising at least one valve disposed between said source of the liquid medium and said conduit, and wherein in said operational mode of said system, said valve is open and allows the liquid medium to flow from said source to said conduit.
4. The system of Embodiment 2, further comprising a pump, wherein in said operational mode of said system, said pump is adapted to pump the liquid medium from said source to said conduit.
5. The system of Embodiment 1, wherein said conduit is a sleeve, adapted to be disposed about a pipe, said pipe adapted to be in fluid communication with a source of the liquid medium in an operational mode of said system.
6. The system of any one of Embodiments 1 to 5, wherein said electromagnetic field generator comprises a metal wire forming a spiral about said conduit, and adapted to generate said electromagnetic field when electricity passes through said metal wire.
7. The system of any one of Embodiments 1 to 6, wherein said electromagnetic field generator is adapted to induce an electromagnetic field having a first frequency in the range of 5 Hz to 500 Hz.
8. The system of Embodiment 7, wherein said first frequency is at least 10 Hz.
9. The system of Embodiment 7, wherein said first frequency is at least 20 Hz.
10. The system of Embodiment 7, wherein said first frequency is at least 25 Hz.
11. The system of Embodiment 7, wherein said first frequency is at least 30 Hz.
12. The system of Embodiment 7, wherein said first frequency is at least 35 Hz.
13. The system of Embodiment 7, wherein said first frequency is at least 40 Hz.
14. The system of any one of Embodiments 7 to 13, wherein said first frequency is at most 400 Hz.
15. The system of Embodiment 14, wherein said first frequency is at most 300 Hz.
16. The system of Embodiment 14, wherein said first frequency is at most 250 Hz.
17. The system of Embodiment 14, wherein said first frequency is at most 200 Hz.
18. The system of Embodiment 14, wherein said first frequency is at most 150 Hz.
19. The system of Embodiment 14, wherein said first frequency is at most 120 Hz.
20. The system of Embodiment 14, wherein said first frequency is at most 100 Hz.
21. The system of Embodiment 14, wherein said first frequency is at most 80 Hz.
22. The system of Embodiment 14, wherein said first frequency is at most 70 Hz.
23. The system of Embodiment 14, wherein said first frequency is at most 60 Hz.
24. The system of any one of Embodiments 1 to 23, wherein said electromagnetic field generator is adapted whereby said electromagnetic field is an alternating current (AC) electromagnetic field.
25. The system of any one of Embodiments 1 to 24, wherein said microwave emitter is adapted to emit microwave radiation having a second or microwave frequency in the range of 0.5 GHz to 20 GHz.
26. The system of Embodiment 25, wherein said second or microwave frequency is within the range of 1 GHz to 18 GHz.
27. The system of Embodiment 25, wherein said second or microwave frequency is within the range of 1 GHz to 16 GHz.
28. The system of Embodiment 25, wherein said second or microwave frequency is within the range of 1 GHz to 14 GHz.
29. The system of Embodiment 25, wherein said second or microwave frequency is within the range of 1 GHz to 12 GHz.
30. The system of Embodiment 25, wherein said second or microwave frequency is within the range of 1 GHz to 10 GHz.
31. The system of Embodiment 25, wherein said second or microwave frequency is within the range of 1 GHz to 8 GHz.
32. The system of Embodiment 25, wherein said second or microwave frequency is within the range of 1 GHz to 6 GHz.
33. The system of Embodiment 25, wherein said second or microwave frequency is within the range of 1 GHz to 5 GHz.
33A. The system of Embodiment 25, wherein said second or microwave frequency is within the range of 1 GHz to 4.5 GHz.
34. The system of Embodiment 25, wherein said second or microwave frequency is within the range of 1 GHz to 4 GHz.
35. The system of Embodiment 25, wherein said second or microwave frequency is within the range of 1 GHz to 3 GHz.
36. The system of Embodiment 25, wherein said second or microwave frequency is within the range of 1.25 GHz to 3 GHz.
37. The system of Embodiment 25, wherein said second or microwave frequency is within the range of 1.5 GHz to 3 GHz.
38. The system of Embodiment 25, wherein said second or microwave frequency is within the range of 1.5 GHz to 2.75 GHz.
39. The system of Embodiment 25, wherein said second or microwave frequency is within the range of 1.5 GHz to 3.5 GHz.
40. The system of Embodiment 25, wherein said second or microwave frequency is within the range of 1.75 GHz to 3.5 GHz.
41. The system of any one of Embodiments 1 to 40, wherein said microwave emitter comprises: a microwave generator; and at least one antenna extending from said microwave generator into said conduit.
42. The system of any one of Embodiments 1 to 41, further comprising a controller, functionally associated with said microwave emitter and with said electromagnetic field generator, said controller being adapted to electronically control or modulate operation of said electromagnetic field generator and of said microwave emitter, in accordance with at least one criterion of said conduit or of the liquid medium within said conduit.
43. The system of Embodiment 42, wherein said at least one criterion includes a flow rate of said liquid medium within said conduit.
44. The system of Embodiment 43, wherein said flow rate is in the range of 1 cm per second to 10 cm per second.
45. The system of Embodiment 43 or 44, wherein a treatment hydraulic retention time of the liquid medium is in the range of 1 second to 120 seconds.
46. The system of Embodiment 45, wherein the treatment hydraulic retention time is at least 10 seconds.
47. The system of Embodiment 45, wherein the treatment hydraulic retention time is at least 25 seconds.
47A. The system of Embodiment 45, wherein the treatment hydraulic retention time is at least 30 seconds.
48. The system of any one of Embodiments 45 to 47, wherein the treatment hydraulic retention time is at most 90 seconds.
48A. The system of Embodiment 48, wherein the treatment hydraulic retention time is at most 75 seconds.
48B. The system of Embodiment 48, wherein the treatment hydraulic retention time is at most 60 seconds.
49. The system of any one of Embodiments 42 to 48, wherein said at least one criterion includes a temperature of said liquid medium within said conduit.
50. The system of any one of Embodiments 42 to 49, wherein said at least one criterion includes a temperature of said conduit.
51. The system of any one of Embodiments 42 to 50, wherein said at least one criterion includes a pH of said liquid medium within said conduit.
52. The system of any one of Embodiments 42 to 51, further comprising at least one sensor, functionally associated with said controller, and adapted to provide to said controller input relating to said at least one criterion.
52A. The system of Embodiment 52, wherein said at least one sensor includes a flow rate sensor.
52B. The system of Embodiment 52 or 52A, wherein said at least one sensor includes a temperature sensor.
52C. The system of any one of Embodiments 52 to 52B, wherein said at least one sensor includes a pH sensor.
53. The system of any one of Embodiments 52 to 53, wherein said at least one sensor includes a chemical sensor.
54. The system of any one of Embodiments 1 to 53, wherein said conduit has a length in the range of 1 m to 5 m.
55. The system of Embodiment 54, wherein said length of said conduit is in the range of 1 m to 4 m.
56. The system of Embodiment 54, wherein said length of said conduit is in the range of 1.5 m to 3 m.
57. The system of any one of Embodiments 1 to 54, wherein said length of said conduit is in the range of 1.75 to 2.25 m.
58. The system of any one of Embodiments 1 to 57, wherein said conduit has an inside diameter in the range of 75 mm to 250 mm.
59. The system of Embodiment 58, wherein said inside diameter of said conduit is in the range of 75 mm to 200 mm.
60. The system of Embodiment 58, wherein said inside diameter of said conduit is in the range of 75 mm to 150 mm.
61. The system of Embodiment 58, wherein said inside diameter of said conduit is in the range of 100 mm to 250 mm.
62. The system of Embodiment 58, wherein said inside diameter of said conduit is in the range of 100 mm to 200 mm.
63. The system of Embodiment 58, wherein said inside diameter of said conduit is in the range of 100 mm to 150 mm.
64. The system of any one of Embodiments 1 to 63, wherein said electromagnetic field generator is disposed closer to an upstream end of said conduit than said microwave emitter, such that, when the liquid medium flows through the conduit, the electromagnetic field is applied to the liquid medium prior to said microwave radiation being applied to the liquid medium.
65. The system of any one of Embodiments 1 to 63, wherein said electromagnetic field generator is disposed closer to a downstream end of said conduit than said microwave emitter, such that, when the liquid medium flows through the conduit, the electromagnetic field is applied to the liquid medium following said microwave radiation being applied to the liquid medium.
66. The system of any one of Embodiments 1 to 63, wherein said electromagnetic field generator is configured to induce said electromagnetic field in a segment said conduit, and said microwave emitter is adapted to emit microwave radiation into said segment of said conduit, such that, when the liquid medium flows through said segment of said conduit, the liquid medium concurrently has said electromagnetic field and said microwave radiation applied thereto.
67. The system of any one of Embodiments 1 to 66, wherein an output of said conduit is in fluid communication with a digester, such that following flowing through said conduit, the liquid medium reaches said digester.
68. The system of Embodiment 67, wherein said digester is an anaerobic digester.
69. The system of Embodiment 67, wherein said digester is an aerobic digester.
70. The system of any one of Embodiments 1 to 69, further comprising the liquid medium including the organic substrate.
71. The system of Embodiment 70, wherein, during passage through said conduit, a temperature of said liquid medium is in the range of 5 C to 60 C.
72. The system of Embodiment 70, wherein, during passage through said conduit, a temperature of said liquid medium is in the range of 15 C to 60 C.
73. The system of Embodiment 70, wherein, during passage through said conduit, a temperature of said liquid medium is in the range of 30 C to 55 C.
74. The system of any one of Embodiments 70 to 73, wherein, during passage through said conduit, a pH of said liquid medium is in the range of 3 to 11.
75. The system of any one of Embodiments 70 to 73, wherein, during passage through said conduit, a pH of said liquid medium is in the range of 4 to 10.
76. The system of any one of Embodiments 70 to 73, wherein, during passage through said conduit, a pH of said liquid medium is in the range of 5 to 10.
77. The system of any one of Embodiments 70 to 73, wherein, during passage through said conduit, a pH of said liquid medium is in the range of 6 to 9.
78. The system of any one of Embodiments 70 to 77, wherein the concentration of said organic substrate within said stream is within the range of 1% to 20%.
79. The system of Embodiment 78, wherein the concentration of said organic substrate within said stream is at least 2.5%.
80. The system of Embodiment 78, wherein the concentration of said organic substrate within said stream is at least 3.5%.
81. The system of Embodiment 78, wherein the concentration of said organic substrate within said stream is at least 4%.
82. The system of any one of Embodiments 78 to 81, wherein the concentration of said organic substrate within said stream is at most 16%.
83. The system of any one of Embodiments 78 to 81, wherein the concentration of said organic substrate within said stream is at most 14%.
84. The system of any one of Embodiments 78 to 81, wherein the concentration of said organic substrate within said stream is at most 12%.
85. The system of any one of Embodiments 78 to 81, wherein the concentration of said organic substrate within said stream is at most 10%.
86. The system of any one of Embodiments 78 to 81, wherein the concentration of said organic substrate within said stream is at most 8%.
87. The system of any one of Embodiments 78 to 81, wherein the concentration of said organic substrate within said stream is at most 7%.
88. The system of Embodiment 78, wherein the concentration of said organic substrate within said stream is within the range of 3% to 15%.
89. The system of Embodiment 78, wherein the concentration of said organic substrate within said stream is within the range of 3% to 12%.
90. The system of Embodiment 78, wherein the concentration of said organic substrate within said stream is within the range of 5% to 12%.
91. The system of Embodiment 78, wherein the concentration of said organic substrate within said stream is within the range of 5% to 10%.
92. The system of any one of Embodiments 70 to 91, wherein said organic substrate comprises biomass.
93. The system of any one of Embodiments 70 to 92, wherein said organic substrate comprises complex organic polymers.
94. The system of Embodiment 93, wherein said complex organic polymers comprise lignin.
95. The system of Embodiment 93 or 94, wherein said complex organic polymers comprise cellulose.
96. The system of any one of Embodiments 93 to 95, wherein said complex organic polymers comprise pectin.
97. The system of any one of Embodiments 70 to 96, wherein said organic substrate comprises industrial waste.
98. The system of any one of Embodiments 60 to 96, wherein said organic substrate comprises sanitary waste.
99. The system of any one of Embodiments 60 to 96, wherein said organic substrate comprises agricultural waste.
100. The system of any one of Embodiments 1 to 99, wherein the energy utilized by said electromagnetic field generator and said microwave emitter to treat a cubic meter of the liquid medium is in the range of 1 kwh to 18 kwh.
101. The system of Embodiment 100, wherein said energy is in the range of 1 kwh to 16 kwh.
101A. The system of Embodiment 100, wherein said energy is in the range of 1 kwh to 14 kwh.
101B. The system of Embodiment 100, wherein said energy is in the range of 1 kwh to 12 kwh.
101C. The system of Embodiment 100, wherein said energy is in the range of 1 kwh to 10 kwh.
101D. The system of Embodiment 100, wherein said energy is in the range of 2 kwh to 10 kwh.
101E. The system of any one of Embodiments 1 to 101D, adapted such that the electromagnetic flux density is at most 6 mT.
101F. The system of Embodiment 101E, adapted such that the electromagnetic flux density is at most 4 mT.
101G. The system of Embodiment 101E, adapted such that the electromagnetic flux density is at most 3.5 mT.
101H. The system of Embodiment 101E, adapted such that the electromagnetic flux density is at most 3 mT.
102. The system of Embodiment 101E, adapted such that the electromagnetic flux density is at most 2.5 mT.
103. The system of any one of Embodiments 101E to 102, wherein adapted such that the electromagnetic flux density is at least 1 mT.
104. The system of Embodiment 103, adapted such that the electromagnetic flux density is at least 1.5 mT.
105. The system of Embodiment 103, adapted such that the electromagnetic flux density is at least 1.75 mT.
106. A method for pre-treating an organic substrate in a liquid medium to facilitate anaerobic or aerobic digestion thereof, the method comprising: allowing a stream of the liquid medium including the organic substrate, to flow through a conduit; and during passage of the liquid medium through said conduit: inducing an electromagnetic field to the interior of said conduit, such that said electromagnetic field is applied to the liquid medium; and emitting microwave radiation into said conduit, such that said microwave radiation is applied to the liquid medium.
107. A method of producing biogas from an organic substrate in a liquid medium, the method comprising: allowing a stream of the liquid medium including the organic substrate, to flow through a conduit; during passage of the liquid medium through said conduit: inducing an electromagnetic field to the interior of said conduit, such that said electromagnetic field is applied to the liquid medium; and emitting microwave radiation into said conduit, such that said microwave radiation is applied to the liquid medium; and producing biogas by digestion of the liquid medium exiting said conduit, following the liquid medium having had said electromagnetic field and said microwave radiation applied thereto.
108. The method of Embodiment 106 or 107, wherein said allowing the liquid medium to flow through said conduit comprises opening a valve between said conduit and a source of the liquid medium to allow the liquid medium to flow into said conduit.
109. The method of Embodiment 106 or 107, wherein said allowing the liquid medium to flow through said conduit comprises pumping the liquid medium into said conduit.
110. The method of any one of Embodiments 106 to 109, wherein said inducing said electromagnetic field comprises passing electricity through a metal wire forming a spiral about said conduit.
111. The method of any one of Embodiments 106 to 110, wherein said electromagnetic field has a first frequency in any one of the ranges of Embodiments 7 to 23.
112. The method of any one of Embodiments 106 to 111, wherein said inducing said electromagnetic field comprises inducing an alternating current (AC) electromagnetic field.
113. The method of any one of Embodiments 106 to 112, wherein said microwave radiation has a second or microwave frequency in the range of any one of the ranges of Embodiments 25 to 40.
114. The method of any one of Embodiments 106 to 113, further comprising electronically controlling or modulating said inducing of said electromagnetic field generator and said emitting of said microwave radiation, in accordance with at least one criterion of the conduit or of the liquid medium within the conduit.
115. The method of Embodiment 114, wherein said at least one criterion includes a flow rate of said liquid medium within said conduit.
116. The method of Embodiment 114, wherein said at least one criterion includes a temperature of said liquid medium within said conduit.
117. The method of Embodiment 114, wherein said at least one criterion includes a temperature of said conduit.
118. The method of Embodiment 114, wherein said at least one criterion includes a pH of said liquid medium within said conduit.
119. The method of any one of Embodiments 114 to 118, wherein said controlling comprises determining whether said at least one criterion is met based on at least one sensor input.
120 The method of Embodiment 119, wherein said at least one sensor input includes input from a flow rate sensor.
121. The method of Embodiment 119 or 120, wherein said at least one sensor input includes input from a temperature sensor.
122. The method of any one of Embodiments 119 to 121, wherein said at least one sensor input includes input from a pH sensor.
123. The method of any one of Embodiments 119 to 122, wherein said at least one sensor input includes input from a chemical sensor.
124. The method of any one of Embodiments 106 to 123, wherein said inducing said electromagnetic field occurs prior to said emitting said microwave radiation, such that, when the liquid medium flows through the conduit, the electromagnetic field is applied to the liquid medium prior to said microwave radiation being applied to the liquid medium.
125. The method of any one of Embodiments 106 to 123, wherein said inducing said electromagnetic field occurs following said emitting said microwave radiation, such that, when the liquid medium flows through the conduit, the electromagnetic field is applied to the liquid medium following said microwave radiation being applied to the liquid medium.
126. The method of any one of Embodiments 106 to 123, wherein said inducing said electromagnetic field occurs concurrently with said emitting said microwave radiation, such that, when the liquid medium flows through said segment of said conduit, the liquid medium concurrently has said electromagnetic field and said microwave radiation applied thereto.
127. The method of any one of Embodiments 106 to 126, further comprising delivering the output of said conduit to an anaerobic digester for anaerobic digestion of the organic substrate in the liquid medium.
128. The method of any one of Embodiments 106 to 126, further comprising delivering the output of said conduit to an aerobic digester for aerobic digestion of the organic substrate in the liquid medium.
129. The method of any one of Embodiments 106 to 128, wherein a temperature of said liquid medium is in the range of 5 C to 60 C.
130. The method of Embodiment 129, wherein said temperature is in the range of 15 C to 60 C.
130. The method of Embodiment 129, wherein said temperature is in the range of 30 C to 55 C.
131. The method of any one of Embodiments 106 to 130, wherein, during passage through said conduit, a pH of said liquid medium is in the range of 3 to 11.
132. The method of any one of Embodiments 106 to 130, wherein, during passage through said conduit, a pH of said liquid medium is in the range of 4 to 10.
133. The method of any one of Embodiments 106 to 130, wherein, during passage through said conduit, a pH of said liquid medium is in the range of 5 to 10.
134. The method of any one of Embodiments 106 to 130, wherein, during passage through said conduit, a pH of said liquid medium is in the range of 6 to 9.
135. The method of any one of Embodiments 106 to 134, wherein the concentration of said organic substrate with said stream is within the range of 1% to 20%.
136. The method of Embodiment 135, wherein the concentration of said organic substrate within said stream is at least 2.5%.
137. The method of Embodiment 135, wherein the concentration of said organic substrate within said stream is at least 3.5%.
138. The method of Embodiment 135, wherein the concentration of said organic substrate within said stream is at least 4%.
139. The method of any one of Embodiments 135 to 138, wherein the concentration of said organic substrate within said stream is at most 16%.
140. The method of any one of Embodiments 135 to 139, wherein the concentration of said organic substrate within said stream is at most 14%.
141. The method of any one of Embodiments 135 to 138, wherein the concentration of said organic substrate within said stream is at most 12%.
142. The method of any one of Embodiments 135 to 138, wherein the concentration of said organic substrate within said stream is at most 10%.
143. The method of any one of Embodiments 135 to 138, wherein the concentration of said organic substrate within said stream is at most 8%.
144. The method of any one of Embodiments 135 to 138, wherein the concentration of said organic substrate within said stream is at most 7%.
145. The method of Embodiment 135, wherein the concentration of said organic substrate within said stream is within the range of 3% to 15%.
146. The method of Embodiment 135, wherein the concentration of said organic substrate within said stream is within the range of 3% to 12%.
147. The method of Embodiment 135, wherein the concentration of said organic substrate within said stream is within the range of 5% to 12%.
148. The method of Embodiment 135, wherein the concentration of said organic substrate within said stream is within the range of 5% to 10%.
149. The method of any one of Embodiments 106 to 148, wherein said organic substrate comprises biomass.
150. The method of any one of Embodiments 106 to 149, wherein said organic substrate comprises complex organic polymers.
151. The method of Embodiment 150, wherein said complex organic polymers comprise lignin.
152. The method of Embodiment 150 or 151, wherein said complex organic polymers comprise cellulose.
153. The method of any one of Embodiments 150 to 152, wherein said complex organic polymers comprise pectin.
154. The method of any one of Embodiments 106 to 153, wherein said organic substrate comprises industrial waste.
155. The method of any one of Embodiments 106 to 154, wherein said organic substrate comprises sanitary waste.
156. The method of any one of Embodiments 106 to 155, wherein said organic substrate comprises agricultural waste.
157. The method of any one of Embodiments 106 to 156, wherein said organic substrate comprises biosolids.
158. The method of any one of Embodiments 106 to 157, wherein the energy utilized during said allowing, said inducing, and said emitting, to treat a cubic meter of the liquid medium, is in the range of 1 kwh to 18 kwh.
159. The method of Embodiment 157, the energy being in the range of 1 kwh to 16 kwh.
160. The method of Embodiment 157, the energy being in the range of 1 kwh to 14 kwh.
161. The method of Embodiment 157, the energy being in the range of 1 kwh to 12 kwh.
162. The method of Embodiment 157, the energy being in the range of 1 kwh to 10 kwh.
163. The method of Embodiment 157, the energy being in the range of 2 kwh to 10 kwh.
164. The method of any one of the previous Embodiments, wherein the electromagnetic flux density of the electromagnetic field is at most 6 millitesla (mT) or at most 5 mT.
165. The system of Embodiment 164, wherein the electromagnetic flux density is at most 4 mT.
166. The system of Embodiment 164, wherein the electromagnetic flux density is at most 3.5 mT.
167. The system of Embodiment 164, wherein the electromagnetic flux density is at most 3 mT.
168. The system of Embodiment 164, wherein the electromagnetic flux density is at most 2.5 mT.
169. The system of any one of Embodiments 164 to 168, wherein the electromagnetic flux density is at least 1 mT.
170. The system of Embodiment 169, wherein the electromagnetic flux density is at least 1.5 mT.
171. The system of Embodiment 169, wherein the electromagnetic flux density is at least 1.75 mT.
172. The method of any one of Embodiments 106 to 171, further comprising any of the features of Embodiments 1 to 105.
173. A method of retrofitting an anaerobic digestion plant to pre-treat organic substrate to be anaerobically digested, the anaerobic digestion plant including an input conduit leading the organic substrate to an anaerobic digester, the method comprising: providing the system for improving digestion of an organic substrate in a liquid medium according to Embodiment 5; and placing said sleeve about the input conduit of the anaerobic digestion plant.
174. A method of retrofitting an aerobic wastewater treatment plant to pre-treat biosolids to increase its bio-degradability, the aerobic wastewater treatment plant including an input conduit leading the organic substrate to an aerobic digester, the method comprising: providing the system for improving digestion of an organic substrate in a liquid medium according to Embodiment 5; and placing said sleeve about said input conduit of said aerobic wastewater treatment plant.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for improving digestion of an organic substrate in a liquid medium, the system comprising:
a conduit, adapted to have a stream flow therethrough, said stream containing the liquid medium and the organic substrate;
an electromagnetic field generator, disposed circumferentially about said conduit, said electromagnetic field generator adapted to induce an electromagnetic field to an interior of said conduit, such that, when the liquid medium flows through said conduit, said electromagnetic field is applied to said liquid medium; and
a microwave emitter, attached to said conduit, said microwave emitter adapted to emit microwave radiation into said conduit, such that, when the liquid medium flows through said conduit, said microwave radiation is applied to the liquid medium;
wherein said microwave emitter is adapted to emit microwave radiation having a microwave frequency in the range of 1 to 4 GHz.

2. The system of claim 1, wherein said electromagnetic field generator is adapted whereby said electromagnetic field is an alternating current (AC) electromagnetic field having a first frequency in the range of 30 Hz to 120 Hz.

3. The system of claim 1 or 2, wherein said microwave emitter comprises: a microwave generator; and at least one antenna extending from said microwave generator into said conduit.

4. The system of any one of claims 1 to 3, further comprising a controller, functionally associated with said microwave emitter and with said electromagnetic field generator, said controller being adapted to electronically control or modulate operation of said electromagnetic field generator and of said microwave emitter, in accordance with at least one criterion of said conduit or of the liquid medium within said conduit.

5. The system of any one of claims 1 to 4, wherein said electromagnetic field generator is configured to induce said electromagnetic field in a segment said conduit, and said microwave emitter is adapted to emit microwave radiation into said segment of said conduit, such that, when the liquid medium flows through said segment of said conduit, the liquid medium concurrently has said electromagnetic field and said microwave radiation applied thereto.

6. The system of any one of claims 1 to 5, wherein an output of said conduit is in fluid communication with a digester, such that following flowing through said conduit, the liquid medium reaches said digester.

7. A method for pre-treating an organic substrate in a liquid medium to facilitate anaerobic or aerobic digestion thereof, the method comprising:
providing the system of any one of claims 1 to 6;
allowing a stream of the liquid medium including the organic substrate, to flow through said conduit; and
during passage of the liquid medium through said conduit:
inducing said electromagnetic field to the interior of said conduit, such that said electromagnetic field is applied to the liquid medium; and
emitting said microwave radiation into said conduit, such that said microwave radiation is applied to the liquid medium; and
producing biogas by digestion of the liquid medium exiting said conduit.

8. The method of claim 7, wherein the electromagnetic flux density of said electromagnetic field is within a range of 1 to 4 millitesla (mT).

9. The method of claim 8, wherein the electromagnetic flux density of said electromagnetic field is at most 3.5 mT.

10. The method of claim 8, wherein the electromagnetic flux density of said electromagnetic field is at most 3 mT.

11. The method of any one of claims 7 to 10, whereby said electromagnetic field is an alternating current (AC) electromagnetic field having a first frequency in the range of 30 Hz to 120 Hz.

12. The method of any one of claims 7 to 11, further comprising electronically controlling or modulating said inducing of said electromagnetic field generator and said emitting of said microwave radiation, in accordance with at least one criterion of the conduit or of the liquid medium within the conduit.

13. The method of claim 12, wherein said at least one criterion includes a flow rate of said liquid medium within said conduit.

14. The method of claim 12 or 13, wherein said at least one criterion includes a temperature of said liquid medium within said conduit.

15. The method of any one of claims 12 to 14, wherein said at least one criterion includes a temperature of said conduit.

16. The method of any one of claims 12 to 15, wherein said at least one criterion includes a pH of said liquid medium within said conduit.

17. The method of any one of claims 12 to 16, wherein said controlling comprises determining whether said at least one criterion is met based on at least one sensor input.

18. The method of any one of claims 12 to 17, wherein said organic substrate comprises biomass.
